(19) United States
(12) Patent Application Publication
(10) Pub. No.: US 2012/0053246 A1
(43) Pub. Date: Mar. 1, 2012

(54) PURIFICATION PROCESS FOR PREPARING HIGHLY PURE ARFORMOTEROL TARTRATE SUBSTANTIALLY FREE OF DESFORMYL IMPURITY

(75) Inventors: Anant Kishanrao Jagtap, Maharashtra (IN); Nandkumar Gaikwad, Maharashtra (IN); Nikhil Trivedi, Maharashtra (IN); Nitin Sharadchandra Pradhan, Maharashtra (IN)

(73) Assignee: ACTAVIS GROUP PTC EHF, Hafnarfjordur (IS)

(21) Appl. No.: 13/215,614

(22) Filed: Aug. 23, 2011

(30) Foreign Application Priority Data
Aug. 26, 2010 (IN) 2469/CHE/2010

(51) Int. Cl. A61K 31/167; C07C 233/43; C07C 231/24

(52) U.S. Cl. 514/630; 562/580; 562/585

(57) ABSTRACT

Provided herein is a highly pure arformoterol tartrate or an amorphous form thereof substantially free of desformyl impurity, 2-hydroxy-5-[(1R)-1-hydroxy-2-[[(1R)-2-(4-methoxyphenyl)-1-methylethyl]amino]ethyl]aniline, process for the preparation thereof, and pharmaceutical compositions comprising the highly pure arformoterol tartrate substantially free of the desformyl impurity.

### CROSS REFERENCE TO RELATED APPLICATION

This application claims the benefit of priority to Indian provisional application No. 2469/CHE/2010, filed on Aug. 26, 2010, which is incorporated herein by reference in its entirety.

### FIELD OF THE DISCLOSURE

The present disclosure relates to a purification process for preparing highly pure arformoterol tartrate substantially free of desformyl impurity, 2-hydroxy-5-[(1R)-1-hydroxy-2-[[(1R)-2-(4-methoxyphenyl)-1-methylethyl]amino]ethyl]aniline.

### BACKGROUND

U.S. Pat. No. 3,994,974 discloses a variety of α-aminomethylbenzyl alcohol derivatives, processes for their preparation, pharmaceutical compositions comprising the derivatives, and methods of use thereof. These compounds have utility as β-adrenergic stimulants and thus have great activity on respiratory smooth muscle and are suitable as bronchodilating agents. Among them, Formoterol, (±)-N-[2-hydroxy-5-[1-hydroxy-2-[[2-(4-methoxyphenyl)-1-methylethyl]amino]ethyl]phenyl]formamide, is a highly potent and [β₂-selective adrenoceptor agonist having a long lasting bronchodilating effect when inhaled. Formoterol is represented by the following structural formula:

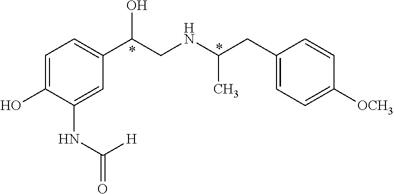

Formoterol has two chiral centers in the molecule, each of which can exist in two possible configurations. This gives rise to four combinations: (R,R), (S,S), (R,S) and (S,R). (R,R) and (S,S) are mirror images of each other and are therefore enantiomers; (R,S) and (S,R) are similarly an enantiomeric pair. The mirror images of (R,R) and (S,S) are not, however, superimposable on (R,S) and (S,R), which are diastereomers. The order of potency of the isomers is (R,R)>>(R,S)═(S,R)>(S,S), and the (R,R)-isomer is 1000-fold more potent than the (S,S)-isomer. Administration of the pure (R,R)-isomer also offers an improved therapeutic ratio.

Various processes for the preparation of formoterol, its enantiomers and related compounds, and their pharmaceutically acceptable salts are disclosed in U.S. Pat. Nos. 3,994,974; 5,434,304; 6,268,533 and 6,472,563; Chem. Pharm. Bull. 26, 1123-1129 (1978); Chirality 3, 443-450 (1991); Drugs of the Future 2006, 31(11), 944-952; and PCT Publication No. WO 2008/035380A2.

The syntheses of all four isomers of formoterol have been reported in the literature, Chem. Pharm. Bull. 26, 1123-1129 (1978) (hereinafter referred to as the ‘CPB Journal’), and Chirality 3, 443-450 (1991) (hereinafter referred to as the ‘Chirality Journal’). In the CPB Journal, the (R,R)- and (S,S)-isomers were obtained by diastereomeric crystallization of racemic formoterol with tartaric acid. In the Chirality Journal, racemic 4-benzyloxy-3-nitrostyrene oxide is coupled with an optically pure (R,R)— or (S,S)—N-(1-phenylethyl)-N-(1-(p-methoxyphenyl)-2-propyl)amine to give a diastereomeric mixture of formoterol precursors, which are then separated by semipreparative HPLC and transformed to the pure formoterol isomers. Both syntheses suffer from long synthetic procedures and low overall yield which is impractical for large scale production of optically pure (R,R)- or (S,S)-formoterol.

U.S. Pat. No. 6,268,533 (hereinafter referred to as the '533 patent) discloses that the L-(+)-tartrate salt of R,R-formoterol is unexpectedly superior to other salts of R,R-formoterol(arformoterol), being easy to handle, pharmaceutically innocuous and non-hygroscopic. Arformoterol, N-[2-hydroxy-5-[(1R)-1-hydroxy-2-[[(1R)-2-(4-methoxyphenyl)-1-methylethyl]amino]ethyl]phenyl]formamide, is a highly potent and selective β₂-adrenergic bronchodilator. Arformoterol is represented by the following structural formula 1:

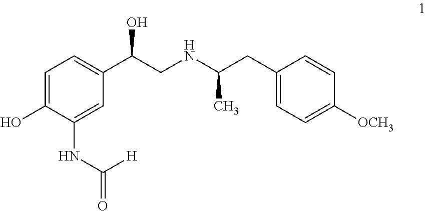

As per the process described in the '533 patent, arformoterol tartrate is prepared by enantioselective reduction of 2-bromo-4′-benzyloxy-3′-nitroacetophenone with borane methyl sulfide in the presence of a chiral oxazaborolidine to produce R-α-(bromomethyl)-4-phenylmethoxy-3-nitrobenzenemethanol; which is then hydrogenated in a Parr hydrogenator in the presence of platinum oxide catalyst to afford the corresponding amino compound; followed by formylation reaction with formic acid in the presence of acetic anhydride to produce (R)—N-[5-(2-bromo-1-hydroxyethyl)-2-(phenylmethoxy)phenyl]formamide; which is then treated with potassium carbonate to produce (R)—N-[5-oxiranyl-2-(phenylmethoxy)phenyl]formamide. The epoxide compound is then condensed with (R)-4-methoxy-α-methyl-N-(phenylmethyl)benzeneethanamine L-mandelate to produce a dibenzyl protected compound, which is then hydrogenated in the presence of a palladium on carbon catalyst to produce arformoterol, followed by reaction with L-tartaric acid to produce arformoterol tartrate.

The '533 patent further discloses that the L-(+)-tartrate salt of arformoterol exists in two polymorphic forms and are designated as crystal Form P1 and crystal Form P2. The '533 patent also describes the conversion of crystal Form P2 to the thermodynamically most stable crystal Form P1.

Five different synthetic routes for preparing arformoterol and its tartrate salt have been reported in the Drugs of the Future 2006, 31(11), 944-952 (hereinafter referred to as the ‘DOF article’). According to first synthetic process, arformoterol tartrate is prepared by resolution of racemic formoterol (I) with D-(+)-tartaric acid. Analogously, racemic p-methoxy-α-methylphenethylamine (II) is resolved by means of D-(+)-tartaric acid to provide the (R)-amine compound (III), which is used as the starting material for the asymmetric synthesis of arformoterol tartrate.

According to second synthetic process as reported in the DOF article, arformoterol is prepared by condensation of (p-methoxyphenyl)acetone (IV) with 1(R)-phenylethylamine (V); followed by diastereoselective hydrogenation of the intermediate imine over Raney nickel to produce the (R,R)-amine (VI); which is then reacted with the racemic epoxide (VII) to produce the amino alcohol adduct (VIII) as an epimeric mixture; followed by subsequent nitro group reduction and formylation in the presence of formic acid and Raney nickel to produce the corresponding mixture of epimeric formamides (IX) and (X); which are separated utilizing semi-preparative chromatography. The desired isomer (X) is finally deprotected by hydrogenation over Pd/C.

According to third synthetic process as reported in the DOF article, arformoterol is prepared by condensation of (R)-4-benzyloxy-3-nitro-styrenoxide (XVII) with (R)-4-methoxy-α-methyl-N-(phenylmethyl)benzeneethanamine (XII) to produce the desired (R,R)-amino alcohol (XVIII); followed by nitro group reduction and subsequent formylation of the resulting amine (XIX) to produce the formamide compound (XX); which is then converted to arformoterol by catalytic hydrogenolysis of its benzyl protecting groups. The amine compound (XII) is prepared by reductive amination of (p-methoxyphenyl)acetone (IV) with benzylamine using H₂ and Pt/C to produce the racemic secondary amine (XI), which is resolved using (S)-mandelic acid to give the optically pure (R)-amine (XII). The (R)-4-benzyloxy-3-nitro-styrenoxide (XVII) is in turn prepared by enantioselective reduction of 2-bromo-4′-benzyloxy-3′-nitroacetophenone (XIII) with borane in the presence of the chiral oxazaborolidines (XVa,b) to give the (R)-bromohydrin (XVI), which is converted to the epoxide (XVII) in the presence of an aqueous base.

The fourth synthetic process as reported in the DOF article, involves a chemoenzymatic approach.

According to fifth synthetic process as reported in the DOF article, arformoterol is prepared by reduction of the enantiopure bromohydrin (XVI) at the nitro group by catalytic hydrogenation. The resulting amine (XXXII) is then converted to formamide (XXXIII), followed by condensation with an amine compound (XII) to produce the amino alcohol (XX), which is finally deprotected by hydrogenation over Pd/C to produce arformoterol.

The processes described in the '533 patent suffer from drawbacks since arformoterol tartrate obtained by the processes does not have satisfactory purity, unacceptable amounts of impurities are formed along with arformoterol tartrate.

Two potential impurities of arformoterol tartrate have been disclosed in the U.S. Pat. No. 6,720,453 (hereinafter referred to as the '453 patent). These impurities are characterized as desformoterol impurity (hereinafter referred to as the desformyl impurity or impurity-A) and dehydroxy formoterol impurity (hereinafter referred to as the dehydroxy impurity or impurity-B), and which have the following structural formulae:

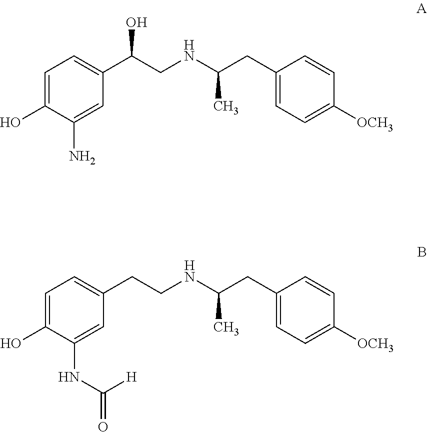

The '453 patent discloses a process for preparing a crystalline solid consisting of greater than 99.5% by weight of arformoterol L-tartrate and less than 0.5% by weight of chemical impurities other than formoterol L-tartrate, wherein the chemical impurities include less than 0.2% by weight of the desformoterol impurity (impurity-A) and/or 0.05% by weight or less of the dehydroxy impurity (impurity-B), and wherein the arformoterol L-tartrate is at least 95% in the polymorphic form of a thermodynamically stable third polymorph A. The process comprises the steps of: 1) formation of a slurry of crude polymorph B by addition of L-tartaric acid to a solution of the arformoterol free base; 2) in-situ conversion of form B to the highly pure form C; 3) isolation of crude polymorph C; 4) dissolution of form C in 50% aqueous isopropyl alcohol (50-55° C.); 5) immediate seeding of the solution with form A crystals (insoluble at 55° C. in 50% aqueous isopropyl alcohol); 6) addition of isopropyl alcohol to decrease the water content to 25% and effect a rapid cooling of the mixture to 40-45° C.; and 7) cooling and isolating the polymorph A of arformoterol tartrate.

According to the '453 patent, the polymorph A is referred to in the '533 patent as P1 and the Polymorph B is referred to in the '533 patent as P2. The '453 patent further teaches that the material (arformoterol L-tartrate) produced according to the processes described in the '533 patent, even in its chemically purest state, contains from 0.2 to 1.5% by weight of chemical impurities, one of which is desformoterol L-tartrate (i.e. impurity-A), and the arformoterol L-tartrate cannot be purified to contain less than 0.2% by weight of any impurity, except by the process of the '453 patent, employing the previously undescribed polymorph C.

However, the processes to produce pure arformoterol tartrate described in the '453 patent also suffer from disadvantages since the processes involve lengthy and cumbersome procedures such as the use of additional solvents, multiple isolation steps, strict control of temperature and time and seeding techniques, and thus resulting in low overall yields of the product. The main drawback of the '453 patent is that the level of desformyl impurity (impurity-A) rises by 0.08%, even though the level of this impurity is reduced initially to 0.04% by warming the slurry of the crude product at 45-50° C. prior to the recrystallization step. Upon recrystallization, 0.12% of desformyl impurity remains in the final product, and which could not be reduced further to less than 0.1% or less than 0.05%.

It is known that synthetic compounds can contain extraneous compounds or impurities resulting from their synthesis or degradation. The impurities can be unreacted starting materials, by-products of the reaction, products of side reactions, or degradation products. Generally, impurities in an active pharmaceutical ingredient (API) may arise from degradation of the API itself, or during the preparation of the API. Impurities in arformoterol or any active pharmaceutical ingredient (API) are undesirable and might be harmful.

Regulatory authorities worldwide require that drug manufacturers isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product is analyzed for purity, typically, by HPLC, TLC or GC analysis, to determine if it is suitable for continued processing and, ultimately, for use in a pharmaceutical product. Purity standards are set with the intention of ensuring that an API is as free of impurities as possible, and, thus, are as safe as possible for clinical use. The United States Food and Drug Administration guidelines recommend that the amounts of some impurities are limited to less than 0.1 percent.

Generally, impurities are identified spectroscopically and by other physical methods, and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRT”) to identify impurities. The RRT of an impurity is its retention time divided by the retention time of a reference marker.

It is known by those skilled in the art, the management of process impurities is greatly enhanced by understanding their chemical structures and synthetic pathways, and by identifying the parameters that influence the amount of impurities in the final product.

There is a need for highly pure arformoterol tartrate substantially free of impurities, as well as processes for preparing thereof.

### SUMMARY

In one aspect, encompassed herein is a process for preparing highly pure arformoterol tartrate comprising desformyl impurity, 2-hydroxy-5-[(1R)-1-hydroxy-2-[[(1R)-2-(4-methoxyphenyl)-1-methylethyl]amino]ethyl]aniline, in an amount of less than about 0.1 area-%, and specifically in an amount of about 0.01 area-% to about 0.05 area-%, as measured by HPLC.

In another aspect, encompassed herein is a process for preparing a highly pure amorphous form of arformoterol tartrate comprising the desformyl impurity in an amount of less than about 0.1 area-%, specifically in an amount of about 0.01 area-% to about 0.05 area-%, as measured by HPLC.

In yet another aspect, provided herein is a highly pure amorphous form of arformoterol tartrate comprising the desformyl impurity in an amount of less than about 0.1 area-%, specifically in an amount of about 0.01 area-% to about 0.05 area-%, as measured by HPLC.

In another aspect, provided herein is a pharmaceutical composition comprising highly pure amorphous form of arformoterol tartrate comprising the desformyl impurity in an amount of less than about 0.1 area-%, specifically in an amount of about 0.01 area-% to about 0.05 area-% (measured by HPLC), and one or more pharmaceutically acceptable excipients.

In still another aspect, provided herein is a pharmaceutical composition comprising a highly pure amorphous form of arformoterol tartrate comprising the desformyl impurity in an amount of less than about 0.1 area-% (measured by HPLC) made by the process disclosed herein, and one or more pharmaceutically acceptable excipients.

In a still further aspect, encompassed is a process for preparing a pharmaceutical formulation comprising combining a highly pure amorphous form of arformoterol tartrate comprising the desformyl impurity in an amount of less than about 0.1 area-% (measured by HPLC) with one or more pharmaceutically acceptable excipients.

In another aspect, the highly pure amorphous form of arformoterol tartrate comprising the desformyl impurity in an amount of less than about 0.1 area-% (measured by HPLC) disclosed herein for use in the pharmaceutical compositions has a D₉₀ particle size of less than or equal to about 500 microns, specifically about 1 micron to about 300 microns, and most specifically about 10 microns to about 150 microns.

### DETAILED DESCRIPTION

Extensive research and experimentation was carried out by the present inventors to reduce the level of the desformyl impurity in arformoterol tartrate. As a result, it has been found that the level of desformyl impurity in arformoterol tartrate can be reduced to below 0.1%, specifically in an amount of about 0.01 area-% to about 0.05 area-% (measured by HPLC), by contacting crude arformoterol tartrate with a mixed anhydride, prepared by the reaction of formic acid and acetic anhydride, in a suitable solvent under appropriate reaction conditions, and isolating highly pure arformoterol tartrate substantially free of desformyl impurity.

According to one aspect, there is provided a highly pure arformoterol tartrate comprising the desformyl impurity, 2-hydroxy-5-[(1R)-1-hydroxy-2-[[(1R)-2-(4-methoxyphenyl)-1-methylethyl]amino]ethyl]aniline, in an amount of less than about 0.1 area-%, and specifically in an amount of about 0.01 area-% to about 0.05 area-%, as measured by HPLC.

In one embodiment, the highly pure arformoterol tartrate disclosed herein has a total purity of greater than about 99.5%, specifically greater than about 99.6%, more specifically greater than about 99.9%, and most specifically greater than about 99.95% as measured by HPLC. For example, the purity of the arformoterol tartrate is about 99.5% to about 99.99%.

According to another aspect, there is provided a purification process for obtaining highly pure arformoterol tartrate comprising a desformyl impurity in an amount of less than about 0.1 area-% as measured by HPLC, comprising:
a) combining crude arformoterol tartrate with a hot aqueous isopropyl alcohol solution at a temperature of above 60° C. to produce a hot reaction mass;
b) filtering the hot reaction mass, followed by washing with isopropyl alcohol to produce a first filtrate;
c) cooling the first filtrate obtained in step-(b) at a temperature of below about 35° C. to produce a second filtrate;
d) combining the second filtrate with a mixed anhydride to produce a reaction mass, wherein the mixed anhydride is prepared by the reaction of formic acid and acetic anhydride, at a temperature of below about 35° C.;
e) recovering highly pure arformoterol tartrate comprising desformyl impurity in an amount of less than about 0.1 area-% from the reaction mass obtained in step-(d); and
(f) optionally converting the highly pure arformoterol tartrate into an amorphous form of arformoterol tartrate.

In one embodiment, the highly pure arformoterol tartrate or amorphous form of arformoterol tartrate obtained by the process disclosed herein comprises the desformyl impurity in an amount of about 0.01 area-% to about 0.05 area-% as measured by HPLC.

Combining of the crude arformoterol tartrate with hot aqueous isopropyl alcohol solution in step-(a) is done in a suitable order, for example, the crude arformoterol tartrate is added to the hot aqueous isopropyl alcohol solution, or alternatively, the hot aqueous isopropyl alcohol solution is added to the crude arformoterol tartrate. The addition is, for example, carried out drop wise or in one portion or in more than one portion. The addition is specifically carried out at a temperature of about 60° C. to about 80° C., and more specifically at a temperature of about 65° C. to about 75° C. After completion of addition process, the resulting mass is stirred at a temperature of about 60° C. to about 80° C. for at least 5 minutes, and more specifically at about 65° C. to about 75° C. for about 10 minutes to about 5 hours.

In one embodiment, the solvent used in the step-(a) is 50% aqueous isopropyl alcohol.

In another embodiment, the first filtrate in step-(c) is cooled at a temperature of about 20° C. to about 35° C., and more specifically at about 25° C. to about 35° C.

Combining of the second filtrate with the mixed anhydride in step-(d) is done in a suitable order as described above. The addition is, for example, carried out drop wise or in one portion or in more than one portion. The addition is specifically carried out at a temperature of about 20° C. to about 35° C., and more specifically at a temperature of about 25° C. to about 30° C. After completion of addition process, the resulting mass is stirred at a temperature of about 20° C. to about 35° C. for at least 1 hour, and more specifically at about 25° C. to about 30° C. for about 3 hours to about 8 hours.

In one embodiment, the mixed anhydride in step-(d) is prepared by stirring the mixture of formic acid and acetic anhydride for at least 10 minutes at a temperature of below about 35° C., and specifically for about 30 minutes to about 2 hours at a temperature of about 20° C. to about 30° C.

The recovery of highly pure arformoterol tartrate comprising the desformyl impurity in an amount of less than about 0.1 area-% obtained in step-(e) is accomplished by techniques such as filtration, filtration under vacuum, decantation, centrifugation, or a combination thereof. In one embodiment, the highly pure arformoterol tartrate is recovered by filtration employing a filtration media of, for example, a silica gel or celite.

The amorphous form of arformoterol tartrate in step-(e) can be prepared in high purity by using the highly pure arformoterol tartrate substantially free of impurities obtained by the method disclosed herein, by known methods, or by the methods disclosed hereinafter.

The highly pure arformoterol tartrate having desformyl impurity in an amount of less than about 0.1 area-% obtained by the above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

According to another aspect, there is provided a highly pure amorphous form of arformoterol L-(+)-tartrate having desformyl impurity in an amount of less than about 0.1 area-%, specifically in an amount of about 0.01 area-% to about 0.05 area-%, as measured by HPLC.

According to another aspect, there is provided a process for preparing highly pure amorphous form of arformoterol L-(+)-tartrate having a desformyl impurity in an amount of less than about 0.1 area-% (as measured by HPLC), comprising:
a) providing a solution comprising highly pure arformoterol L-(+)-tartrate having desformyl impurity in an amount of less than about 0.1 area-% and a solvent, wherein the solvent is an organic solvent or a solvent medium comprising water and an organic solvent, wherein the organic solvent is selected from the group consisting of an alcohol, a ketone, a hydrocarbon, a chlorinated hydrocarbon, and mixtures thereof;
b) optionally, filtering the solution to remove insoluble matter; and
c) substantially removing the solvent from the solution to provide the highly pure amorphous form of arformoterol L-(+)-tartrate having the desformyl impurity in an amount of less than about 0.1 area-%, wherein the removal of the solvent is accomplished by distillation or complete evaporation of the solvent, spray drying, vacuum drying, lyophilization or freeze drying, agitated thin-film (ATFD) drying, or a combination thereof.

In one embodiment, the solvent used in step-(a) is selected from the group consisting of water, methanol, ethanol, isopropyl alcohol, n-butanol, tert-butanol, acetone, methyl ethyl ketone, methyl isobutyl ketone, methyl tert-butyl ketone, n-hexane, n-heptane, cyclohexane, toluene, methylene chloride, and mixtures thereof.

Specifically, the solvent is selected from the group consisting of water, methanol, ethanol, isopropyl alcohol, and mixtures thereof; and a most specific solvent is aqueous methanol.

In another embodiment, the removal of the solvent in step-(c) is accomplished by spray drying, in which a solution of arformoterol L-(+)-tartrate is sprayed into the spray drier at the flow rate ranging from 10 to 300 ml/hr, specifically 40 to 200 ml/hr. The air inlet temperature to the spray drier used may range from about 30° C. to about 150° C., specifically from about 65° C. to about 110° C. and the outlet air temperature used may range from about 30° C. to about 90° C.

The highly pure amorphous form of arformoterol tartrate obtained by the above process may be further dried as per the methods described hereinabove.

Further encompassed herein is the use of the highly pure amorphous form of arformoterol L-(+)-tartrate having the desformyl impurity in an amount of less than about 0.1 area-%, specifically in an amount of about 0.01 area-% to about 0.05 area-%, for the manufacture of a pharmaceutical composition together with a pharmaceutically acceptable carrier.

A specific pharmaceutical composition of highly pure amorphous form of arformoterol L-(+)-tartrate having the desformyl impurity in an amount of about 0.01 area-% to about 0.1 area-% is selected from a solid dosage form and an oral suspension.

In one embodiment, the highly pure amorphous form of arformoterol L-(+)-tartrate having the desformyl impurity in an amount of less than about 0.1 area-%, specifically in an amount of about 0.01 area-% to about 0.05 area-%, has a D₉₀ particle size of less than or equal to about 500 microns, specifically about 1 micron to about 300 microns, and most specifically about 10 microns to about 150 microns.

In another embodiment, the particle sizes of the highly pure amorphous form of arformoterol L-(+)-tartrate having the desformyl impurity in an amount of less than to about 0.1 area-% are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided pharmaceutical compositions comprising highly pure amorphous form of arformoterol L-(+)-tartrate having the desformyl impurity in an amount of less than about 0.1 area-% prepared according to the process disclosed herein and one or more pharmaceutically acceptable excipients.

According to another aspect, there is provided a process for preparing a pharmaceutical formulation comprising combining highly pure amorphous form of arformoterol L-(+)-tartrate having the desformyl impurity in an amount of less than about 0.1 area-% prepared according to the process disclosed herein, with one or more pharmaceutically acceptable excipients.

Yet in another embodiment, pharmaceutical compositions comprise at least a therapeutically effective amount of highly pure amorphous form of arformoterol L-(+)-tartrate having the desformyl impurity in an amount of less than about 0.1 area-%. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The highly pure amorphous form of arformoterol L-(+)-tartrate having the desformyl impurity in an amount of less than about 0.1 area-% may also be administered as ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described herein.

### Experimental Details:

### HPLC Method for Measuring Chemical Purity:

The purity was measured by high performance liquid chromatography under the following conditions:
Apparatus: Water's HPLC system having alliance 2695 model pump and 2487 (UV) detector with Empower chromatography software or its equivalent.

### Chromatographic Conditions:

Column: Symmetry C 18, (75×4.6 mm), 3.5 μMake: Waters, Part No: WAT066224
Detector wavelength: UV at 214 nm
Flow rate: 1.5 mL/min
Sample cooler temperature: 4° C.
Injection volume: 20.0 μL
Column oven temperature: 35° C.
Run time: 45 minutes
Elution: Gradient
   Diluent buffer preparation (pH 6.0):
Weigh and transfer about 6.10 g of Sodium dihydrogen phosphate monohydrate and 1.03 g of Di-sodium hydrogen phosphate dihydrate in 1000 mL of water, sonicate to dissolve and check the pH of the dilution. The pH should be 6.00 (±0.10).
   Diluent Preparation: Mix 840 mL of the above buffer with 160 mL of Acetonitrile. Mobile phase buffer preparation:
Weigh and transfer about 9.10 g of Potassium dihydrogen phosphate anhydrous and 5.0 g of 1-Octane sulfonic acid sodium salt in 1000 mL water, adjust the pH to 3.0 (±0.05) using dilute orthophosphoric acid solution. Filter through 0.22 μ porosity membrane and degas.
Mobile phase—A: Buffer (100%)
Mobile phase—B: Buffer: Acetonitrile (50:50) (v/v)

### Gradient Program:

| | Mobile phase - A | Mobile phase - B |
|---|---|---|
| Time (min) | (%) | (%) |
| 0 | 50 | 50 |
| 5 | 50 | 50 |
| 18 | 20 | 80 |
| 38 | 20 | 80 |
| 39 | 50 | 50 |
| 45 | 50 | 50 |

### Gas Chromatography (GC):

Gas chromatograph (Agilent 6890N GC system equipped with flame ionization detector or its equivalent). Head space sampler (Head space G1888 or its equivalent). Data handling system (Waters Empower or its equivalent).
Column: DB-624; 30 meter length, 0.53 mm internal diameter, 1.8 μm film thickness, fused silica capillary column
Column Temp.: 40° C. (hold for 5 minutes) to 60° C. @ 2° C./minute (hold for 0 minute) to 120° C. @ 6° C./minute (hold for 5 minutes) to 200° C. @ 20° C./minute for 6 minutes.
Injector/detector: 200° C./260° C. Carrier gas: Nitrogen @ 1.0 mL/min, linear velocity
Split Ratio: (5:1)

### Head Space Parameters:

Oven Temperature: 80° C.
Needle temperature: 90° C.
Transfer line temperature: 100° C.
GC cycle time: 50.0 minutes
Thermostatic time: 30.0 minutes
Pressurization time: 0.5 minute
Loop fill time: 0.5 minute
Loop equilibrium time: 0.2 minute
Injection time: 1.0 minute

The following examples are given for the purpose of illustrating the present disclosure and should not be considered as limitation on the scope or spirit of the invention.

### COMPARATIVE EXAMPLE 1

Preparation of pure (R,R)-Formoterol L-tartrate (Arformoterol tartrate) as per the prior art process disclosed in U.S. Pat. No. 6,268,533.

A mixture of isopropyl alcohol (75 ml) and water (75 ml) was heated at 65-70° C., followed by the addition of crude arformoterol tartrate (25 g, HPLC purity: 98%, and content of desformyl impurity: 0.3%) under stirring at 65-70° C. The mixture was stirred for 5-6 minutes, followed by hot filtration and then washing with isopropyl alcohol (12.5 ml). The resulting filtrate was cooled to 30-35° C. and stirred for 5 hours at 25-30° C. The resulting mass was isolated by filtration, followed by drying at 50-60° C. to give 21 g of pure N-[2-hydroxy-5-[(1R)-1-hydroxy-2-[[(1R)-2-(4-methoxyphenyl)-1-methylethyl]amino]ethyl]phenyl]formamide (2R,3R)-2,3-dihydroxybutane dioate (1:1) (Arformoterol tartrate) [HPLC purity: 99.5%; Enantiomeric purity: 99.8%; Desformyl impurity: 0.25%, [α]²⁰_{D}=−30° (C=0.61%, water)].
Observation: During the purification of arformoterol tartrate (using IPA:Water solvent mixture), the content of the desformyl impurity increases with number of purifications.

### COMPARATIVE EXAMPLE 2

Preparation of pure (R,R)-Formoterol L-tartrate (Arformoterol tartrate) as per the prior art process disclosed in Chem. Pharm. Bull. 26(4), pages 1123-1129 (1978)

A mixture of isopropyl alcohol (80 ml) and water (20 ml) was heated at 65-70° C., followed by the addition of crude arformoterol tartrate (10.0 g, obtained by the process reported in the above mentioned C.P.B journal). The mixture was stirred for 5-6 minutes, followed by cooling at 30-35° C. and stirring for overnight at 25-30° C. The resulting mass was isolated by filtration, followed by drying at 50-60° C. to give 4.3 g of arformoterol tartrate [HPLC Purity: 95.10%; Desformyl impurity: 0.16%].

### EXAMPLES

### Example 1

Preparation of N-[5-[(1R)-Hydroxy-2-[[(1R)-methyl-2-(4-methoxyphenyl)ethyl](phenyl methyl)amino]ethyl]-2-(phenylmethoxy)phenyl]-formamide Polyethylene glycol-400 (90 ml) was added to (R,R)-3-amino-α-[[[2-(4-methoxyphenyl)-1-methylethyl](phenylmethyl)amino]methyl]-4-(phenylmethoxy)-benzenemethanol (45 g) under stirring at 20-30° C., followed by the addition of formic acid (90 ml). The resulting mass was heated to 50-55° C. for 4-5 hours. The reaction mass was further cooled to 20-30° C., followed by quenching with water (450 ml) and adjusting the pH to 7-7.5 using 10% sodium bicarbonate solution (2 L). The reaction mass was then extracted two times with ethyl acetate (2×150 ml). The ethyl acetate layer was washed with water (450 ml) and dried over anhydrous sodium sulfate (50 g) and then the solvent was distilled out to give 46 g of N-[5-[(1R)-hydroxy-2-[[(1R)-methyl-2-(4-methoxyphenyl)ethyl](phenylmethyl)amino]ethyl]-2-(phenylmethoxy)phenyl]-formamide [HPLC purity: 98%; enantiomeric purity: 99%; [α]²⁰_{D}=−90° to −100° (C=1, chloroform).

### Example 2

### Preparation of (R,R)-Formoterol (Arformoterol)

A solution of N-[5-[(1R)-hydroxy-2-[[(1R)-methyl-2-(4-methoxyphenyl)ethyl](phenylmethyl)amino]ethyl]-2-(phenylmethoxy)phenyl]-formamide (20 g) in ethanol (200 ml) was hydrogenated in a hydrogenation flask by applying 1-2 kg pressure in the presence of Pd/C catalyst (10% Pd, 50% wet, 10 g), until the completion of the reaction. The reaction mixture was filtered through celite and washed with methanol (20 ml), followed by distillation of methanol on rotavapour under reduced pressure to give 12.5 g of arformoterol base as amorphous solid [HPLC purity 98%; enantiomeric purity 98.5%; Desformyl impurity 0.45%; [α]²⁰_{D}=−40° (C=0.53%, chloroform).

### Example 3

### Preparation of Crude (R,R)-Formoterol L-tartrate (Arformoterol tartrate Crude)

Isopropyl alcohol (150 ml) and a solution of L-tartaric acid (21.8 g) in water (150 ml) were added to (R,R)-formoterol base (50 g) under stirring at 20-30° C. The mixture was stirred for 5-6 hours at 25-30° C. and filtered, washed with isopropyl alcohol (150 ml) and then dried at 50° C. to produce 62.5 g of crude arformoterol tartrate. [HPLC purity 98%; enantiomeric purity 98.5%; Desformyl impurity 0.3%; and [α]²⁰_{D}=−28° (C=0.61%, water).

### Example 4

### Preparation of Pure (R,R)-Formoterol L-tartrate (Arformoterol tartrate)

A mixture of isopropyl alcohol (150 ml) and water (150 ml) was heated at 65-70° C., followed by the addition of crude arformoterol tartrate (50 g, obtained in example 3) under stirring at 65-70° C. The mixture was stirred for 5-6 minutes, and then the resulting hot reaction mass was filtered and washed with isopropyl alcohol (25 ml). The resulting filtrate was cooled to 30-35° C. Meanwhile, a mixed anhydride was prepared by stirring formic acid (0.5 ml) and acetic anhydride (1 ml) for 30 minutes at 25-30° C. The mixed anhydride was added to the above cooled filtrate and then stirred for 5 hours at 25-30° C. The resulting mass was isolated by filtration, followed by drying at 50-60° C. to give 43 g of pure N-[2-hydroxy-5-[(1R)-1-hydroxy-2-[[(1R)-2-(4-methoxyphenyl)-1-methylethyl]amino]ethyl]phenyl]formamide (2R,3R)-2,3-dihydroxybutanedioate (1:1) (Arformoterol tartrate) [HPLC purity: 99.88%; Enantiomeric purity: 99.98%; Desformyl impurity 0.03%, [α]²⁰_{D}=−30° (C=0.61%, water)].

### Example 5

### Preparation of Amorphous form of Arformoterol tartrate

Arformoterol tartrate (50 g, obtained in example 4) was added to a mixture of methanol (1000 ml) and water (1000 ml), followed by stirring to provide a clear solution. The clear solution was spray dried under the conditions given below to produce 20 g of amorphous arformoterol tartrate [HPLC purity: 99.6%; Enantiomeric purity: 99.98%; Desformyl impurity 0.03%, [α]²⁰_{D}=−30° (0.61%, water)].
Conditions for Spray Drying: Aspirator=70 mbar; Feed rate=20-30% (400-500 ml/hr);
Inlet temperature=140-145° C.; Outlet temperature=65-80° C.; Atomizer air pressure=1.5-2 kg; and Vacuum=−220 to −150 mm
Level of organic volatile impurities: Methanol: 450 parts per million (ppm); isopropyl alcohol: 200 ppm; dichloromethane: Below detection limit (BDL); and ethanol: BDL.

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP).

The term “crude arformoterol tartrate” as used herein refers to arformoterol tartrate containing desformyl impurity in an amount of greater than about 0.1 area-%, specifically about 0.1 area-% to about 0.5 area-%%, as measured by HPLC.

The term “micronization” used herein means a process or method by which the size of a population of particles is reduced.

As used herein, the term “micron” or “μm” both are equivalent refers to “micrometer” which is 1×10⁻⁶ meter.

As used herein, “crystalline particles” means any combination of single crystals, aggregates and agglomerates.

As used herein, “Particle Size Distribution (PSD)” means the cumulative volume size distribution of equivalent spherical diameters as determined by laser diffraction in Malvern Master Sizer 2000 equipment or its equivalent.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

We claim:

1. A purification process for obtaining highly pure arformoterol tartrate comprising desformyl impurity in an amount of less than about 0.1 area-% as measured by HPLC, comprising:
a) combining crude arformoterol tartrate with a hot aqueous isopropyl alcohol solution at a temperature of above 60° C. to produce a hot reaction mass;
b) filtering the hot reaction mass, followed by washing with isopropyl alcohol to produce a first filtrate;
c) cooling the first filtrate obtained in step-(b) at a temperature of below about 35° C. to produce a second filtrate;
d) combining the second filtrate with a mixed anhydride to produce a reaction mass, wherein the mixed anhydride is prepared by reaction of formic acid and acetic anhydride at a temperature of below about 35° C.;
e) recovering highly pure arformoterol tartrate comprising the desformyl impurity in an amount of less than about 0.1 area-% from the reaction mass obtained in step-(d); and
f) optionally converting the highly pure arformoterol tartrate into an amorphous form of arformoterol tartrate.

2. The process of claim 1, wherein the arformoterol tartrate or the amorphous form of arformoterol tartrate obtained has the desformyl impurity in an amount of about 0.01 area-% to about 0.05 area-% as measured by HPLC.

3. The process of claim 1, wherein the solvent used in the step-(a) is 50% aqueous isopropyl alcohol.

4. The process of claim 1, wherein the reaction mass obtained in step-(a) is stirred at a temperature of about 60° C. to about 80° C.; wherein the first filtrate obtained in step-(c) is cooled at a temperature of about 20° C. to about 35° C.; wherein the reaction mass obtained in step-(d) is stirred at a temperature of about 20° C. to about 35° C. for at least 1 hour; and wherein the mixed anhydride in step-(d) is prepared by stirring the mixture of formic acid and acetic anhydride for at least 10 minutes at a temperature of below about 35° C.

5. The process of claim 4, wherein the reaction mass obtained in step-(a) is stirred at a temperature of about 65° C. to about 75° C. for about 10 minutes to about 5 hours;
wherein the first filtrate obtained in step-(c) is cooled at a temperature of about 25° C. to about 35° C.; wherein the reaction mass obtained in step-(d) is stirred at a temperature of about 25° C. to about 30° C. for about 3 hours to about 8 hours; and wherein the mixed anhydride in step-(d) is prepared by stirring the mixture of formic acid and acetic anhydride for about 30 minutes to about 2 hours at a temperature of about 20° C. to about 30° C.

6. The process of claim 1, wherein the recovery of highly pure arformoterol tartrate comprising desformyl impurity in an amount of less than about 0.1 area-% obtained in step-(e) is accomplished by filtration, filtration under vacuum, decantation, centrifugation, filtration employing a filtration media of a silica gel or celite, or a combination thereof.

7. The process of claim 1, wherein the conversion of the highly pure arformoterol tartrate into the amorphous form of arformoterol tartrate in step-(e) is carried out by a process comprising:
a) providing a solution comprising highly pure arformoterol L-(+)-tartrate having the desformyl impurity in an amount of less than about 0.1 area-% and a solvent, wherein the solvent is an organic solvent or a solvent medium comprising water and an organic solvent, wherein the organic solvent is selected from the group consisting of an alcohol, a ketone, a hydrocarbon, a chlorinated hydrocarbon, and mixtures thereof;
b) optionally, filtering the solution to remove insoluble matter; and
c) substantially removing the solvent from the solution to provide the highly pure amorphous form of arformoterol L-(+)-tartrate having the desformyl impurity in an amount of less than about 0.1 area-%, wherein the removal of the solvent is accomplished by distillation or complete evaporation of the solvent, spray drying, vacuum drying, lyophilization, freeze drying, agitated thin-film (ATFD) drying, or a combination thereof.

8. The process of claim 7, wherein the solvent used in step-(a) is selected from the group consisting of water, methanol, ethanol, isopropyl alcohol, and mixtures thereof.

9. The process of claim 8, wherein the solvent used in step-(a) is aqueous methanol.

10. The process of claim 7, wherein the removal of the solvent in step-(c) is accomplished by spray drying.

11. Arformoterol tartrate or an amorphous form thereof comprising a 2-hydroxy-5-[(1R)-1-hydroxy-2-[[(1R)-2-(4-methoxyphenyl)-1-methylethyl]amino]ethyl]aniline (desformyl impurity) in an amount of about 0.01 area-% to about 0.05 area-% as measured by HPLC.

12. A pharmaceutical composition comprising arformoterol tartrate or an amorphous form thereof comprising a 2-hydroxy-5-[(1R)-1-hydroxy-2-[[(1R)-2-(4-methoxyphenyl)-1-methylethyl]amino]ethyl]aniline (desformyl impurity) in an amount of about 0.01 area-% to about 0.05 area-%, and one or more pharmaceutically acceptable excipients.
